(19) United States
(12) Patent Application Publication
(10) Pub. No.: US 2009/0081304 A1
(43) Pub. Date: Mar. 26, 2009

(54) Composite pigment nanoparticles and processes to form organic-inorganic nanoparticle composite particles

(76) Inventors: Lye Han Valerie Choy, Singapore (SG); Shivkumar Chiruvolu, San Jose, CA (US); Nobuyuki Kambe, Menlo Park, CA (US); Vladimir K. Dioumaev, Mountain View, CA (US); Hui Du, Sunnyvale, CA (US); Shinichi Kuramoto, Shizuoka-ken (JP); Hiroto Higuchi, Tokyo (JP)

Correspondence Address: DARDI & ASSOCIATES, PLLC, 220 S. 6TH ST., SUITE 2000, U.S. BANK PLAZA, MINNEAPOLIS, MN, 55402, US

(21) Appl. No.: 11/903,522

(22) Filed: Sep. 21, 2007

(51) Int. Cl. A61K 9/14; C09B 67/20; B02C 23/18

(52) U.S. Cl. 424/490; 106/400; 106/505; 106/499; 524/1; 241/20

(57) ABSTRACT

Milling approaches provide for the efficient formation of composite particles having an inorganic nanoparticle core with an organic coating composition. The nanoparticles can additionally function as a milling media or distinct milling particles can be used and later separated from the product composite particles. In general, the milling is performed in the presence of a dispersing agent that facilitates dispersing of the composite particles in a carrier liquid. The processes described herein can be effectively used in the formation of composite particles comprising organic pigments. Similarly, the composite particles can be formed with other organic compounds, such as organic pharmaceutical compositions.

### FIELD OF THE INVENTION

The invention relates to composite particles formed with an organic composition, such as an organic pigment, and inorganic nanoparticles that are blended to form composite nanoparticles. The invention further relates to dispersions of the composite particles in carrier liquids. Also, the invention relates to mixing or milling approaches for the formation of inorganic particle-organic solid composite particles. The composite particles can be used as pigment particles in some embodiments.

### BACKGROUND OF THE INVENTION

Pigment particles find a wide range of applications with respect to production of commercial products. For example, pigments are incorporated into a wide range of coatings for visual effects, such as paints and the like. A significant growing use of pigments is in the formation of toner particles, in particular for color toners. Pigments also find wide use in inks and the like for a wide range of printing applications as well as in other applications such as color filters.

Inorganic nanoparticles can be provided which have low absorbance of visible light. However, these particles have strong absorption of ultraviolet light. The nanoscale inorganic particles can be used advantageously in applications to take advantage of their low visible absorption and/or their high ultraviolet absorption.

### SUMMARY OF THE INVENTION

In a first aspect, the invention pertains to a dispersion of particles comprising a carrier liquid, a dispersing agent and composite pigment particles. The composite pigment particles generally comprise a blend of an inorganic particle and an organic pigment. The composite pigment particles have a Z-average dispersed particle size in the organic carrier liquid of no more than about 300 nm.

In a further aspect, the invention pertains to a collection of composite particles comprising inorganic core particles and an organic pigment in which the composite particles have an average diameter of no more than about 300 nm. Also, the inorganic core particles can be substantially free of a surface modifying agent bonded between the inorganic core particles and the organic pigment. Generally, the composite particles comprise at least about 5 weight percent organic pigment.

In another aspect, the invention pertains to a method for forming composite pigment particles comprising applying effective mixing conditions to a blend of inorganic particles and organic pigment in an organic carrier liquid with a pigment dispersing agent to form dispersed composite particles with a Z-average particle size of no more than about 300 nm.

In other aspects, the invention pertains to a method for forming composite particles from an organic composition and inorganic nanoparticles in a carrier liquid in which the organic composition and the inorganic particles are substantially insoluble in the carrier liquid. The method comprises applying effective mixing conditions to a combination of the organic composition, a dispersing agent and the inorganic nanoparticles in the carrier liquid without the presence of milling beads, to mill the organic composition onto particles having an average particle size of no more than about 250 nm. In some embodiments, the inorganic nanoparticles have an average particle size of no more than about 200 nm.

Moreover, the invention pertains to a collection of composite particles comprising non-toxic inorganic particles coated with a pharmaceutical composition, in which the inorganic nanoparticles have an average particle size of no more than about 250 nm.

### BRIEF DESCRIPTION OF THE DRAWINGS

FIG. 1 is a schematic diagram indicating the process of forming composite particles from organic particles and inorganic nanoparticles.

FIG. 2 is a schematic view of an enlarged toner particles formed with composite particles in which the composite particle is shown in a partially cut away expanded view.

FIG. 3 is a collection of four optical micrographs, with two micrographs corresponding to samples milled without TiO₂ nanoparticles and two micrographs corresponding to samples milled with TiO₂ nanoparticles and with two magnifications for each sample.

FIG. 4 is a collection of two transmission electron micrographs of composite particles corresponding with sample 3 at two different magnifications.

FIG. 5 is a plot of differential light scattering measurements of particle size distribution for four samples of composite pigment particles.

FIG. 6 is a plot of coloring power for three samples of composite pigment particles.

FIG. 7 is a collection of two transmission electron micrographs of composite pigment particles at two different magnifications showing the organic shell on the inorganic core particles.

FIG. 8 is a plot of optical parameters for six composite pigment samples and a standard sample.

FIG. 9 is a plot of color power as a function of deposited mass for four composite pigment particle samples and a standard sample.

FIG. 10 is a plot of color power as a function of deposited thickness for the four samples used in the plots of FIG. 9.

FIG. 11 is a plot of color power as a function of deposited volume for three samples milled with different amounts of pigment dispersing agent.

FIG. 12 is a plot of color power as a function of deposited mass for six composite pigment particle samples.

FIG. 13 is a plot of color power as a function of deposited volume for the same six samples used for the plots in FIG. 12.

FIG. 14 is a plot of optical parameters for composite pigment samples prepared with different amounts of pigment dispersing agent.

FIG. 15 is a plot of differential light scattering particle size distributions for four samples of composite pigment particles prepared under different milling conditions.

FIG. 16 is a collection of two transmission electron micrographs with the right image showing a sample milled with milling beads and the left image showing a second sample milled without milling beads.

FIG. 17 is a collection of two transmission electron micrographs of other embodiments of composite pigment particles with the right image showing a sample milled with milling beads and the left image showing a sample milled without milling beads.

FIG. 18 is a collection of two transmission electron micrographs of further embodiments of composite pigment particles with the right image showing a sample milled with milling beads and the left image showing a sample milled without milling beads.

FIG. 19 is a plot of color power as a function of deposited mass for eight samples of composite pigment particles.

FIG. 20 is a collection of two transmission electron micrographs taken for another embodiment of composite pigment particles taken at two different magnifications.

FIG. 21 is a plot of differential light scattering measurements of particle size distribution for three samples milled under different conditions.

### DETAILED DESCRIPTION OF THE INVENTION

Composite particles can comprise an inorganic particle core along with an organic composition, such as an organic pigment, coated directly onto the inorganic particles. The composite particles can be formed using a mixing, e.g., milling, process in which organic compositions become coated onto the inorganic particles in the presence of a suitable dispersing agent for the organic composition. In some embodiments, uniform inorganic nanoparticles provide the cores for the formation of the composite particles such that the resulting composite particles can have a submicron average particle size, as well as, good particle uniformity. Using appropriate wet milling approaches described herein, uniform composite particles can be formed without surface modifying the inorganic particles with a surface bound modifying composition. In some embodiments, the inorganic nanoparticles function as milling beads for the milling process such that other milling media is not used.

Pigments are used commercially in a range of products for visualization purposes. Commercial pigments include both organic and inorganic pigments. However, to obtain a broad range of desired colors, organic pigments are generally used, optionally in combination with inorganic pigments. For example, organic pigments can be used in paints and coatings, as well as within molded plastics and the like. In some embodiments, organic pigments are used for the formation of toners, inks and the like. In some embodiments, organic compositions can be coated onto the inorganic particles for other functions besides for use as pigments. For example, organic pharmaceutical compositions can be used to form composite particles to increase surface area.

The composite pigment particles comprise an inorganic particle with organic compositions covering the surface of the inorganic particle. The relative amounts of the components can be selected to achieve desired properties of the product composite particles. For appropriate embodiments, if an organic pigment forms a coating on the inorganic particles, a greater portion of the organic pigment can be available for visualization purposes. In particular, if light is only able to penetrate a certain distance into the organic pigment, using a shell with a distance approaching this penetration depth results in a greater amount of color enhancement for a particular amount of pigment mass. The color properties are a function of the organic pigment surface area, which then becomes a function of the inorganic particle surface area. In this way, the color enhancement performance of the composite can be enhanced for a given amount of organic pigment.

The organic pigment can be effectively coated over a large fraction of the inorganic particle surface. The resulting composite particles can have a layer of organic pigment that is relatively uniformly coated onto the inorganic particles at a selected thickness. The uniform layer of organic pigment on inorganic particles yields composite particles with a size distribution that reflects the size distribution of the starting inorganic particles. The composite particles generally are formed directly in a dispersion, which can have well dispersed composite particles.

Pigments are insoluble compositions that can be dispersed as small particles within an appropriate dispersion. Organic pigments generally can have a selected visible color and generally also can include, for example, organometallic compositions as well as metal free pigments. Colors of the pigments can be black or other color, such as a primary color. In some embodiments, it is desirable to produce different composite pigment particles with individual composite particle collections having a selected pigment color, such as yellow, blue, magenta/red, green and/or the like. As printing technology improves, it may be desirable to have a larger range of available colors to provide the ability of printing with a corresponding greater range of printed colors through the combination of a greater range of printable inks, toners and the like.

The inorganic particles can be selected based on their desired properties. In general, the inorganic particles can have a small average particles size to provide a correspondingly large surface area. The large surface area provides a correspondingly large surface to spread a relatively thin layer of organic pigment. In general, the inorganic particles have an average primary particle size of no more than about 500 nm and in some embodiments no more than about 50 nm. For particles with an average particle size of no more than about 50 nanometers and for many inorganic compositions, the inorganic particles generally have an absorption spectrum in the ultraviolet region of the electromagnetic spectrum.

The properties of the inorganic particles can be selected based on the particular use of the composite particles. If the index or refraction is selected to be a larger value, then the resulting composite has a high index of refraction so that the eventual material scatters light more and has a glossy appearance. If a lower index of refraction material is used, the resulting composite will form a material with a deeper color and with the appearance of less gloss. With respect to optical or other electromagnetic properties of the inorganic particles, the particles can be white to provide better hiding power or phosphors that provide contributions to the color power of the composite. In some embodiments, the inorganic particles can be reflective of infrared light to provide heat resistant composites that result in desired heat management. For pharmaceutical applications the inorganic particles should be non-toxic to humans and in some embodiments, non-toxic to mammals, such as pets and farm animals.

Highly uniform nanoparticles can be produced using laser pyrolysis, but in general particles formed using other techniques are also suitable. Laser pyrolysis is versatile with respect to the production of a wide range of inorganic particle compositions. Other suitable techniques for particle production include, for example, solution based techniques and other flow based techniques besides laser pyrolysis.

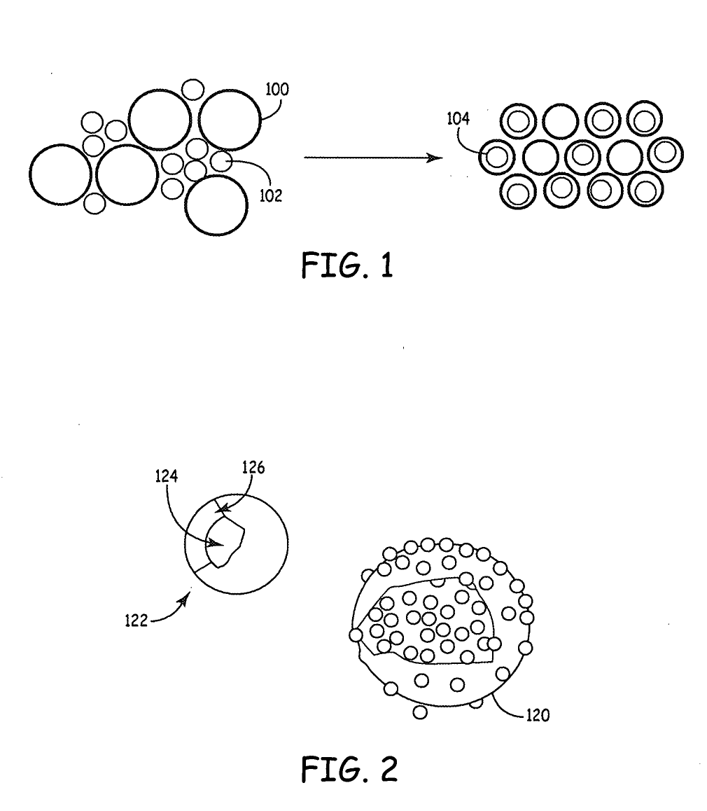

The organic composition can be spread over the inorganic particle surfaces using strong mixing conditions along with a dispersing agent for the organic composition. The process is indicated schematically in FIG. 1 in which the arrow represents the application of strong mixing conditions to a combination of organic particles 100 and inorganic particles 102 to form composite particles 104 or core shell particles in a carrier liquid in the presence of an organic composition dispersing agent. Dispersing agents are known to stabilize organic pigment dispersions. It has been surprisingly found that the use of the pigment dispersing agent removes the need to surface modify the inorganic particles. Strong mixing conditions can be provided through the use of high shear, such as with a mixer or a mill, or through the application of ultrasonic vibration or the like.

In particular, inorganic particles can be interacted with surface modifying agents that covalently or otherwise strongly bind to the surface of the inorganic particles to introduce an organic or organometallic surface coating onto the particles. As discussed below, surface modifying the inorganic particles has been found to be an alternative approach to facilitate the spreading of the organic pigment onto the inorganic particle surfaces. However, the surface modifying compound is an undesirable component of the composite particles from a functional standpoint since it adds weight and cost while performing no advantageous optical or other function in the composite particles. In some embodiments described herein, the present approaches removes at least one step in the process since the inorganic particles are not surface modified prior to forming the composite particles.

The pigment dispersing agent can be substantially recycled as the solvent and/or the resulting dispersion can be carried over into a subsequent processing step in which the pigment dispersing agent can be used to facilitate the formation of a paint, coating composition, ink, toner particle and the like. Thus, while the pigment dispersing agent is an extra composition for use in the processing of the composite particles, the pigment dispersing agent may be used for further processing, in some embodiments.

The dispersing agents interact with the organic compositions to improve the dispersibility of the organic compositions. For organic pigments, the dispersing agents have one or more functional groups that interact with the pigment particles. Thus, the interaction of the dispersing agent with the pigment improves the stability of a dispersion of the pigment. While not wanting to be limited by theory, the dispersing agents generally are believed to inhibit flocculation of the pigment particles since flocculation of the pigment particles is though to lead to settling of the pigment particles from the dispersion. Suitable pigment dispersing agents include, for example, both polymeric and non-polymeric compositions that are soluble in a selected solvent to be used to disperse the pigment. Both ionic and nonionic polymeric pigment dispersing agents are commercially available.

To perform the composite particle formation, a pigment, inorganic particles and a dispersing agent are combined in a carrier liquid. The blend is then subjected to strong mixing conditions. The carrier liquid can be an organic liquid or an aqueous liquid. The dispersing agent is soluble in the carrier liquid and the dispersing agent can be selected to be appropriately soluble in the selected carrier liquid. As demonstrated in the Examples below, the strong mixing conditions are effective to spread the organic pigment over the surface of the inorganic particles.

As noted above, the composite pigment particles are generally suitable for use in paints, coatings, inks, toners and the like. Paints, coatings and inks are generally similar in that the composite pigment particles are dispersed in the product. Since the composite particles have an organic pigment coating, a suitable dispersing agent further facilitates the dispersion of the composite particles in the product liquid. Thus, the dispersing agent used to form the composite particles can be usefully carried over into a liquid product to correspondingly stabilize the dispersion of the composite pigment particles in the product.

The incorporation of composite pigment particles into a toner particle is shown schematically in FIG. 2. Toner particle 120 comprises a plurality of composite pigment particles 122. Composite particles 122 comprise a core inorganic particle 124 with an organic pigment coating 126. The composite pigment particle in general can have a black or other color. Use of color toner is growing at a rapid rate. Based on the teachings herein, the toner particles comprise the composite pigment particles and a resin that has desirable setting properties to fix the toner image.

As noted above, the composite pigment particles can be used to decrease the amount of organic pigment needed for a desired amount of color visualization. This effect is a result of the spreading the organic pigment over a larger surface area. The processing approaches are efficient with respect to processing time as well as resources used in the processing. Also, the elimination of an inorganic particle surface modifier reduces the associated cost, generally eliminates at least one processing step and improves the color power for a given weight of organic pigment since more color is harvested out of thin layers of organic pigment than out of conventional thick particles of organic pigment. The composite pigment particles can be used for other optical applications in addition to toner applications. For example, the composite pigment particles can be blended with a polymer to form a composite film. The composite film can be used as a color filter or the like, which can be selected to have a desired index of refraction through the corresponding selection of the inorganic particles. Thus, the color filters can be incorporated into an optical stack to form a multilayered antireflective or similar coating or the like based on the index of refraction selection while similarly providing desired color filtering through the pigment properties.

Some of the processing techniques described herein can be used for non-pigment applications. In particular, the processing techniques can be used to apply other organic compositions onto inorganic nanoparticles. For example, the milling approaches can be used to apply drugs or the like onto inorganic particles to facilitate delivery of the drugs. In particular, upon application of the drug onto the inorganic particles, the surface area is increased so that sparingly soluble drugs can dissolve more readily upon delivery into a patient in contact with the patient's aqueous bodily fluids.

### Composite Particles

The composite particles comprise an inorganic core with an organic coating, such as an organic pigment coating. The organic pigment generally is selected to have a desired color and/or other visual properties. Spreading the organic pigment onto the inorganic core increases the effective surface area of the organic pigment. The technique can be effective to increase the surface area of other organic compositions also, as well as to provide desirable properties of both the organic composition and the inorganic material within the resulting composite particles.

The organic pigments are softer materials that can be spread over the inorganic particles in the preparation process. In some embodiments, the composite particles comprise from about 5 to about 99.5 weight percent inorganic particles and in further embodiments from about 10 to about 90 weight percent inorganic particles. Similarly, in some embodiments, the composite particles comprise from about 0.5 weight percent organic pigment to about 95 weight percent organic pigment and in further embodiments from about 10 weight percent to about 90 weight percent organic pigment. Generally, the relative amounts of organic pigment and inorganic particles are evaluated based on the amount of starting materials added to the mixing composition. A person of ordinary skill in the art will recognize that additional ranges within the explicit ranges above the relative amounts of organic pigment and inorganic particles are contemplated and are within the present disclosure.

The organic composition, e.g., pigment, generally forms a shell over the inorganic particle core. As described in the Examples below, the organic shell generally can be observed using transmission electron microscopy. In some embodiments, the thickness of the visible organic shell can be from about 1 nm to about 75 nm, in further embodiments from about 2 nm to about 50 nm and in additional embodiments from about 3 nm to about 40 nm. A person of ordinary skill in the art will recognize that additional ranges of shell thickness within the explicit ranges above are contemplated and are within the present disclosure.

In some embodiments of particular interest, the composite particles consist essentially of inorganic particles and organic pigments. In particular, the composite particles generally do not comprise a surface modification agent bound to the inorganic particles, which form a boundary layer between the inorganic particles and the organic pigment. It has surprisingly been found that the organic pigments can be effectively spread onto the inorganic particle surfaces without a surface modifying agent on the inorganic particles. However, the organic pigments can comprise property-modifying agents within the organic pigments or other additives that effectively modify the organic pigment physical properties. Similarly, an organic pigment can comprise a mixture of organic pigment compositions.

For the sake of clarity with respect to the discussion in the previous paragraph, inorganic particle surface modifying agents may chemically bond with the inorganic particle. If the surface modification agent does not chemically bond with the particles, this composition generally associates with the surface due to a plurality of non-specific interactions and/or entropic effects such that the surface modification agent effectively and strongly binds with the inorganic particle surface. Thus, the inorganic particle surface modifying agents are distinct from surfactants and the like, which may have weak interaction with the inorganic particles. Surfactants and other weakly interacting compositions are in equilibrium with the solution and have a majority of the composition in solution.

Suitable organic pigments can be selected and used as the organic pigment in the composite particle depending on desired properties of the composite particles. The organic pigments can comprise inorganic and/or organometallic components and/or functional groups. Suitable pigments generally include, for example, commercially available organic pigments. Examples of a yellow organic pigment include, for example, a monoazo pigment, such as C.I. Pigment Yellow 1 (e.g., Symuler Fast Yellow GH, Dainippon Ink and Chemicals, Inc.) and C.I. Pigment Yellow 74, a disazo pigment, such as C.I. Pigment Yellow 12 (e.g., Symuler Fast Yellow GF, Dainippon Ink and Chemicals, Inc.) and C.I. Pigment Yellow 17 (e.g., Symuler Fast Yellow 8GR, Dainippon Ink and Chemicals, Inc.), a non-benzidine azo pigment, such as C.I. Pigment Yellow 180, an azo lake pigment, such as C.I. Pigment Yellow 100, a condensed azo pigment, such as C.I. Pigment Yellow 95, an acidic dye lake pigment, such as C.I. Pigment Yellow 15, a basic dye lake pigment, such as C.I. Pigment Yellow 18, an anthraquinone pigment, such as Flavanthrone Yellow, an isoindolinone pigment, such as Isoindolinone Yellow 3RLT, a quinophthalone pigment, such as Quinophthalone Yellow, an isoindoline pigment, such as Isoindoline Yellow, a nitroso pigment, such as C.I. Pigment Yellow 153, a metallic complex azomethine pigment, such as C.I. Pigment Yellow 117, and an isoindolinone pigment, such as C.I. Pigment Yellow 139.

Examples of a magenta organic pigment include a monoazo pigment, such as C.I. Pigment Red 3, a disazo pigment, such as C.I. Pigment Red 38, an azo lake pigment, such as C.I. Pigment Red 53:1 and C.I. Pigment Red 57:1, a condensed azo pigment, such as C.I. Pigment Red 144, an acidic dye lake pigment, such as C.I. Pigment Red 174, a basic dye lake pigment, such as C.I. Pigment Red 81, an anthraquinone pigment, such as C.I. Pigment Red 177, a thioindigo pigment, such as C.I. Pigment Red 88, a perynone pigment, such as C.I. Pigment Red 194, a perylene pigment, such as C.I. Pigment Red 149, a quinacridone pigment, such as C.I. Pigment Red 122, an isoindolinone pigment, such as C.I. Pigment Red 180, an alizarin lake pigment, such as C.I. Pigment Red 83, a naphthol pigment, such as pigment Red 269 (PR260), C.I. Pigment Red 57 (e.g., Symuler Brill Carmine LB, Dainippon Ink and Chemicals, Inc.), C.I. Pigment Red 21 (e.g., Sany Fast Red GR, Sanyo Color Works, Ltd.) and C.I. Pigment Red 112 (e.g., Symuler Fast Red FGR, Dainippon Ink and Chemicals, Inc.).

Examples of a cyan organic pigment include a disazo pigment, such as C.I. Pigment Blue 25, a phthalocyanine pigment, such as C.I. Pigment Blue 15 (e.g., Fastogen Blue GS, Dainippon Ink and Chemicals, Inc.), an acidic dye lake pigment, such as C.I. Pigment Blue 24, a basic dye lake pigment, such as C.I. Pigment Blue 1, an anthraquinone pigment, such as C.I. Pigment Blue 60, C.I. Pigment Blue 16 (e.g., Sumitone Cyanine Blue LG, Sumitomo Chemical Co., Ltd.) and an alkali blue pigment, such as C.I. Pigment Blue 18.

In some embodiments, a single pigment composition is used to form the composite particles. However, in some embodiments, a plurality of pigment compositions can be incorporated into particular composite particles. For example, two, three or more organic pigment compositions can be used within a collection of composite particles. Similarly, for embodiments based on other organic compositions, the composite particles can comprise a plurality of organic compositions, such as a plurality of drugs or other biological agents. A drug is considered broadly as any agent that results in a physiological effect upon introduction into a patient.

In general, inorganic particles with any stable composition can be used to form the composites. The composition of the inorganic particles can be selected based on the desired properties of the composite, for example, with respect to index of refraction. Similarly, the physical properties of the inorganic particles, such as primary particle size and uniformity, can be selected by the nature of the desired composite particles with respect to secondary particle size and other physical properties and processing considerations.

Small and uniform inorganic particles can provide processing advantages with respect to forming uniform composite particles. In addition, small inorganic particles have desirable properties for optical applications including, for example, a shifted absorption spectrum toward the ultraviolet and reduced scattering of visible light. Suitable nanoparticles can be formed, for example, by laser pyrolysis, flame synthesis, combustion, or solution-based processes, such as sol gel approaches. In particular, laser pyrolysis is useful in the formation of particles that are highly uniform in composition, crystallinity and size. Laser pyrolysis involves light from an intense light source that drives the reaction to form the particles. Laser pyrolysis is an excellent approach for efficiently producing a wide range of nanoscale particles with a selected composition and a narrow distribution of average particle diameters. Alternatively, submicron particles can be produced using a flame production apparatus such as the apparatus described in U.S. Pat. No. 5,447,708 to Helble et al., entitled “Apparatus for Producing Nanoscale Ceramic Particles,” incorporated herein by reference. Furthermore, submicron particles can be produced with a thermal reaction chamber such as the apparatus described in U.S. Pat. No. 4,842,832 to Inoue et al., entitled “Ultrafine Spherical Particles of Metal Oxide and a Method for the Production Thereof,” incorporated herein by reference.

For convenience, light-based pyrolysis is referred to as laser pyrolysis since this terminology reflects the convenience of lasers as a radiation source, and is a conventional term in the art. Laser pyrolysis approaches discussed herein incorporate a reactant flow that can involve gases, vapors, aerosols or combinations thereof to introduce desired elements into the flow stream. The versatility of generating a reactant stream with gases, vapor and/or aerosol precursors provides for the generation of particles with a wide range of potential compositions. The production of a range of particles by laser pyrolysis is described further in published U.S. Patent Application 2003/203205A to Bi et al., entitled “Nanoparticle Production and Corresponding Structures,” incorporated herein by reference.

A collection of submicron/nanoscale particles may have an average diameter for the primary particles of less than about 500 nm, in some embodiments from about 2 nm to about 100 nm, alternatively from about 2 nm to about 75 nm, or from about 2 nm to about 50 nm. A person of ordinary skill in the art will recognize that other ranges within these specific ranges are covered by the disclosure herein. Primary particle diameters are evaluated by transmission electron microscopy or the alike.

As used herein, the term “particles” refer to physical particles, which are unfused, so that any fused primary particles are considered as an aggregate, i.e. a physical particle. For particles formed by laser pyrolysis, the particles can be generally effectively the same as the primary particles, i.e., the primary structural element within the material. If there is hard fusing of some primary particles, these hard fused primary particles form correspondingly larger physical particles. The primary particles can have a roughly spherical gross appearance, or they can have rod shapes, plate shapes or other non-spherical shapes. Upon closer examination, crystalline particles generally have facets corresponding to the underlying crystal lattice. Amorphous particles generally have a spherical aspect. Diameter measurements on particles with asymmetries are based on an average of length measurements along the principle axes of the particle.

Because of their small size, the particles tend to form loose agglomerates due to van der Waals and other electromagnetic forces between nearby particles. These loose agglomerates can be dispersed in a carrier liquid to a significant degree and in some embodiments approximately completely to form dispersed primary particles. The size of the dispersed particles can be referred to as the secondary particle size. The primary particle size, of course, is the lower limit of the secondary particle size for a particular collection of particles, so that the average secondary particle size can be approximately the average primary particle size if the primary particles are substantially unfused and if the particles are effectively completely dispersed in the carrier liquid.

Even though the particles may form loose agglomerates, the nanometer scale of the particles is clearly observable in transmission electron micrographs of the particles. The particles generally have a surface area corresponding to particles on a nanometer scale as observed in the micrographs. Furthermore, the particles can manifest unique properties due to their small size and large surface area per weight of material. For example, the absorption spectrum of crystalline, nanoscale TiO₂ particles is shifted into the ultraviolet.

The particles can have a high degree of uniformity in size. Laser pyrolysis generally results in particles having a very narrow range of particle diameters. Furthermore, heat processing under suitably mild conditions generally does not significantly alter the very narrow range of particle diameters. With aerosol delivery of reactants for laser pyrolysis, the distribution of particle diameters is particularly sensitive to the reaction conditions. Nevertheless, if the reaction conditions are properly controlled, a very narrow distribution of particle diameters can be obtained with an aerosol delivery system. As determined from examination of transmission electron micrographs, the particles generally have a distribution in sizes such that at least about 95 percent, and in some embodiments 99 percent, of the particles have a diameter greater than about 35 percent of the average diameter and less than about 220 percent of the average diameter. In additional embodiments, the particles generally have a distribution in sizes such that at least about 95 percent, and in some embodiments 99 percent, of the particles have a diameter greater than about 40 percent of the average diameter and less than about 160 percent of the average diameter. In embodiments of particular interest, the particles have a distribution of diameters such that at least about 95 percent, and in some embodiments 99 percent, of the particles have a diameter greater than about 60 percent of the average diameter and less than about 140 percent of the average diameter. A person of ordinary skill in the art will recognize that other ranges of uniformity within these specific ranges are covered by the disclosure herein.

Furthermore, in some embodiments essentially no particles have an average diameter greater than about 5 times the average diameter, in other embodiments about 4 times the average diameter, in further embodiments 3 times the average diameter, and in additional embodiments 2 times the average diameter. In other words, the particle size distribution effectively does not have a tail indicative of a small number of particles with significantly larger sizes. This is a result of the small reaction region to form the inorganic particles and corresponding rapid quench of the inorganic particles. An effective cut off in the tail of the size distribution indicates that there are less than about 1 particle in 10⁶ has a diameter greater than a specified cut off value above the average diameter. High particle uniformity can be exploited in a variety of applications.

In addition, the nanoparticles for incorporation into the composite particles may have a high purity level. Furthermore, crystalline nanoparticles, such as those produced by laser pyrolysis, can have a high degree of crystallinity. Similarly, the crystalline nanoparticles produced by laser pyrolysis can be subsequently heat processed to improve and/or modify the degree of crystallinity and/or the particular crystal structure. Impurities on the surface of the particles may be removed by heating the particles to achieve not only high crystalline purity but high purity overall.

For high index-of-refraction inorganic particles, rutile titanium dioxide is a suitable material. Suitable lower index-of-refraction inorganic particles for optical applications can comprise silicon dioxide. In some embodiments, the inorganic particles can be white to provide good hiding power. For heat resistant composite particles, the inorganic particles can have a composition that reflects infrared light, such as metal particles.

In some embodiments, the inorganic particles can be phosphors. Thus, the phosphor particles are excited through the interaction with an electric field or the like and subsequently emit light at a particular wavelength. Similarly, the inorganic particles can fluoresce or phosphoresce, such that the particles absorb light at one wavelength and emit light at a longer wavelength. For example, the particles can absorb ultraviolet light and emit visible light. Thus, in these embodiments, the inorganic particles can further contribute to the coloring power of the organic pigment through the emission of light from the core-shell particles.

With respect to drug delivery applications, magnetic inorganic particles can facilitate the drug delivery process. In particular, magnetic fields can be used to direct the particles for delivery. The use of magnetic particles for drug delivery is described further, for example, in published PCT application WO 2006/102377A to Akhtari et al., entitled “Functionalized Magnetic Nanoparticles and Method of Use Thereof,” incorporated herein by reference. Suitable compositions for the magnetic particles include, for example, magnetic nickel ferrite, NiFe₂O₄ as well as iron oxides.

The composite particles are also characterized by a secondary particle size. However, secondary particle size is a characteristic of a dispersion. Thus, secondary particle size is described further below in the description of the dispersions.

### Dispersion Compositions and Dispersion Properties

The composite particles are formed in a liquid dispersion having a pigment dispersing agent. The resulting dispersion of composite particles can be characterized with respect to the resulting properties of the composite particles. The liquid generally can comprise an organic liquid and/or an aqueous liquid. The dispersion can be correspondingly used for further processing into an ultimate product or the composite particles can be harvested from the dispersion. Formation of the dispersion and the composite particles is described in the following section.

The composite particles are formed directly in a dispersion with an appropriate liquid present. As used herein, the term dispersion is used very broadly in the sense of a mixture of solid particles with a liquid. The term dispersion does not necessarily refer to good dispersion in the sense of a stable dispersion with dispersed particles in a liquid that do not settle. In particular, the product dispersion may be a good dispersion even though the initial, processing dispersion may not be a good dispersion with respect to one or more component particles. Generally, the particles in the initial dispersion used for processing of the composite particles may not be well dispersed with respect to the inorganic particle and/or the initial organic pigment particles. The desired processing to form the composite particles generally does not depend on the degree of dispersion of the particles in the initial dispersion. For convenience, the initial dispersion can be referred to as a processing dispersion while the dispersion following milling can be referred to as the composite particle dispersion.

The processing dispersions for the formation of composite particles generally comprise inorganic nanoparticles, organic pigment, a carrier liquid and a pigment dispersing agent. The processing dispersion can comprise other processing aids, as appropriate. As discussed further below, milling beads or milling balls may be used in the processing dispersion to facilitate the formation of the composite particles. Inorganic nanoparticles and organic pigments are discussed above. Generally, particles used to facilitate mill are generally referred to as milling media. As used herein, milling balls and milling beads generally have average particle diameters of at least about 10 microns and substantially all milling particles of a milling media have a diameter of at least about 2 microns, as discussed further below.

In some embodiments, the processing dispersion comprises from about 0.5 to about 50 percent by weight, in further embodiments from about 1 to about 40 percent by weight and in other embodiments from about 2 to about 30 percent by weight of the combined weights of the inorganic particles, the organic pigment and the pigment dispersing agent. In addition, the processing dispersion comprises in some embodiments from about 50 to about 99.5 weight percent carrier liquid, in further embodiments from about 60 to about 99 weight percent carrier liquid, and in additional embodiments from about 70 to about 98 weight percent carrier liquid. The processing dispersion generally comprises from about 0.01 to about 100 parts by weight pigment dispersing agent relative to the weight of organic pigment, in further embodiments from about 0.05 to about 25 parts by weight, in other embodiments from about 0.1 to about 10 and in additional embodiments from about 0.20 to about 5 parts by weight pigment dispersing agent relative to one part by weight of the organic pigment. When the dispersing agent content is too low, the effectiveness to stabilize dispersion of the organic pigment may be insufficient. When the dispersing agent content is too high, the pigment might be unnecessarily diluted to a point of having undesirable effect on the coloring power as well as other processing and product-related properties of the resulting composite particle dispersion for some applications, such as the glass transition temperature for toners and other physical and mechanical properties of pigment particle-polymer composites. A person of ordinary skill in the art will recognize that additional ranges of concentration and amounts within the explicit ranges above are contemplated and are within the present disclosure. The relative amounts of inorganic particles and organic pigment generally is selected based on the desired composition of the resulting composite particles as discussed above, since the organic pigment and the inorganic particles in the processing dispersion generally are incorporated into the composite particles during processing.

The use of an effective dispersing agent, e.g., a pigment dispersing agent, provides for surprisingly improved results in the formation of the composite particles. In general, pigment dispersing agents act as stabilizers, inhibiting agglomeration and/or flocculation of dispersed pigment particles. A suitable dispersing agent is soluble in the selected dispersing liquid. Thus, there is an equilibrium between the solubilized dispersing agent and a portion of the solubilized dispersing agent associated with the surface of pigment particles. A dispersing agent can be identified by the ability to form a more stable dispersion relative to an equivalent dispersion formed without the pigment dispersing agent.

For pigment embodiments, the deflocculating effect of the pigment dispersing agent generally results for example, in a significant reduction of the viscosity of a mill base comprising organic pigments. This reduction in the viscosity of mill base can provide for increased loading levels of pigments and significant improvement in milling efficiency. While not wanting to be limited by theory, pigment dispersion stabilization is thought to follow one of at least two different mechanisms: ionic and non-ionic. Ionic dispersing agents can provide dispersion stabilization through an electrostatic stabilization mechanism. This mechanism is based on establishing a charged double layer at the pigment particle/liquid interface. The charged ionic dispersing agent can be adsorbed onto the pigment surface, producing a charged layer with the immediate surrounding liquid and forming a diffused part of the double layer. When other particles approach the diffused parts of the double layer, they begin to interpenetrate, giving rise to strong electrostatic repulsive forces that tend to separate the particles.

Non-ionic dispersing agents can provide a steric stabilization mechanism. This mechanism is also referred to as an entropic stabilization mechanism. The barrier, formed by the molecules on the pigment surface, reduces the attractive forces between pigment particles. Non-ionic dispersing agents, such as the Ciba® EFKA® 4000 series, can be used in the dispersions described herein. The Ciba® EFKA® 4000 series involves high molecular weight dispersing agents, which are linear or branched molecules with a polyurethane or polyacrylate structure. The molecular weights of these polymers are generally between 5000 and 30,000 atomic mass unit, i.e. g/mole. These polymers have pendant anchoring groups, which adsorb onto the surface of the organic pigment particles. A pigment dispersing agent suitable for use in aqueous liquids are described, for example, in U.S. Pat. No. 5,854,323 to Itabashi et al., entitled “Pigment Dispersing Agent, Composition Containing the Same, and Aqueous Pigment Dispersion,” incorporated herein by reference. Monomeric pigment dispersing agents are described, for example, in U.S. Pat. No. 6,790,576 to Fujimoto et al., entitled “Dispersing Agent for Pigment, Pigment-Dispersion Composition, Toner, and Toner Production Process,” and U.S. Pat. No. 7,147,704 to Ueno, entitled “Pigment Dispersing Agent, Pigment Composition and Pigment Dispersion,” both of which are incorporated herein by reference.

The product dispersion comprises composite particles, pigment dispersing agent, and carrier liquid. The general properties of the composite particles are discussed above. Since the composite particles have organic pigment along their surfaces, the pigment dispersing agent stabilizes the dispersion of composite particles. The composite particles are dispersed as secondary particles in the dispersion. Secondary particle size refers to the particle size in the dispersion. While, in principle, the secondary particle size can approach the primary particle size, incomplete dispersion of the particles generally results in a secondary particle size somewhat larger than the primary particle size.

Secondary particle size can be measured, for example, using light scattering in a liquid dispersion. The ability of a liquid to disperse particles within the liquid depends on the surface properties of the particles, the nature of the liquid and additives in the liquid, the concentration of particles and the process used to disperse the particles as well as the physical particle size. In some embodiments, the secondary particles have a Z-average particle size or a volume average particle size of no more than about 1000 nm, in additional embodiments no more than about 500 nm, in further embodiments from about 2 nm to about 300 nm, in other embodiments from about 2 nm to about 100 nm, and alternatively about 2 nm to about 50 nm. A Z-average particle size can be obtained from a dynamic light scattering measurement of a dispersion, as described further in the examples below. A person of ordinary skill in the art will recognize that other ranges within these specific ranges are contemplated and are within the present disclosure.

The distribution of sizes of secondary particles within a liquid dispersion can be measured by established approaches, such as dynamic light scattering. The width of the distribution can be evaluated as the full width at half maximum. Suitable particle size analyzers include, for example, a Microtrac UPA instrument from Honeywell based on dynamic light scattering, a Horiba Particle Size Analyzer from Horiba, Japan and ZetaSizer Series of instruments from Malvern based on Photon Correlation Spectroscopy. The principles of dynamic light scattering for particle size measurements in liquids are well established.

### Processing to Form Composite Particles

It has been surprisingly discovered that an organic pigment can be effectively spread onto the surfaces of inorganic nanoparticles without surface modifying the inorganic particles. The processing of the organic pigment and inorganic particles generally comprises the formulation of a processing dispersion with a polymer dispersing agent. Processing dispersions are described in detail above with respect to composition. The processing dispersion is generally subject to appropriate mixing conditions, such as the application of high shear. The properties of the resulting composite particles are also described in detail above.

In general, the organic pigment particles and the inorganic particles may or may not be separately processed prior to combining the compositions for the formation of the processing dispersion. For example, the organic pigment can be milled prior to formation of the processing dispersion. Similarly, the inorganic particles may be dispersed prior to formation of the processing dispersion. The inorganic particles can be dispersed through the selection of a liquid compatible with the inorganic particle surface chemistry and generally through the use of mild to moderate mixing conditions. The formation of inorganic particle dispersions without particle surface modification is described further in copending U.S. patent application Ser. No. 11/645,084 to Chiruvolu et al., entitled “Composites of Polymers and Metal/Metalloid Oxide Nanoparticles and Methods of Forming These Composites,” incorporated herein by reference. Whether or not preliminary processing of the components is performed, the processing dispersion components are combined to form the processing dispersion.

Then, to process the processing dispersion to form the composite particles, appropriate mixing conditions are applied to the processing dispersion. Mixing conditions can be applied through the use of shear, kneading, sonication or the like. Various mixing apparatuses are designed for the application of shear. For example, milling can be performed in a bead mill, a ball mill, jet milling, blenders, other mixing apparatuses or the like. Suitable mills are commercially available. Alternatively, kneaders or spatula stroking apparatuses can be used to provide the mixing. In general, the degree of mixing can be evaluated empirically with respect to the success in spreading the organic composition onto the inorganic particles. The appropriate mixing conditions may depend on the particular solid organic composition and the liquid.

If separate milling particles are used, these generally have an average diameter of at least about 10 microns, in further embodiments at least about 25 microns and in other embodiments from about 30 microns to about 5 mm. The terminology generally is used with larger milling particles being referred to as balls while smaller milling particles being referred to as beads, although the terminology is not particularly significant. Similarly, the milling particles generally have less than 1 particle in 1×10⁵ particles with a particle size less than about 2 microns. A person of ordinary skill in the art will recognize that additional ranges within the explicit ranges above are contemplated and are within the present disclosure. The milling particles are selected to not have many if any small particles so that the milling particles can be separated form the dispersion following processing. The milling particles can be separated from the dispersion using filtration, centrifugation or the like.

In general, the milling can be performed with or without separate milling particles. It has been surprisingly discovered that significant milling with respect to organic compositions can be performed using the inorganic nanoparticles themselves as the milling media. Thus, the inorganic nanoparticles can be effective at reducing the size of organic particles as well as with respect to coating the inorganic nanoparticles with an appropriate organic composition.

This processing of organic solids using the inorganic nanoparticles can be effective with other organic solids besides organic pigments. For example, suitable organic pharmaceutical compositions can be milled in this way. In general, pharmaceutical compositions are compositions that have a medicinal effect on human or other mammals, such as a pet or a farm animal. Suitable pharmaceutical compositions include, for example, analgesics, antibiotics, anti-cancer agents and the like. In some embodiments, the pharmaceutical compositions are substantially insoluble in aqueous liquids so that the materials can be milled in an aqueous liquid. If the pharmaceutical compositions are substantially insoluble in aqueous liquids, it can be useful to mill the particles to form composite particles to increase the surface area of the organic composition and to increase the speed of uptake into the patient's bodily fluids.

Suitable dispersing agents can be selected based on the particular organic compositions. See, for example, Published U.S. Patent Application 2004/0110667 to Linn, entitled “Carrier System for Cyclosporine Pharmaceutical Compositions,” incorporated herein by reference. The composite particles comprising a pharmaceutical organic composition can be incorporated into a suitable formulation for delivery to a patient. For example, if the composite particles are dispersed into a suitable aqueous liquid, such as buffered saline, the liquid can be delivered by ingestion, injection or the like. Similarly, the composite particles can be processed into tablets, capsules or other convenient formulation.

With the application of shear, suitable rpm generally range from about 5 to about 10,000 and in further embodiments from about 20 to about 5000. To obtain comparable strong mixing conditions using ultrasound, suitable powers generally range from about 10 W to about 20,000 W and in further embodiments from about 50 W to about 16,000 W. A person of ordinary skill in the art will recognize that additional ranges of mixing parameters within the explicit ranges above are contemplated and are within the present disclosure. In general, suitable shear can depend on the configuration of the mixing vessel and the total volume being processed, and similarly suitable sonication powers can depend on the mixing vessel and the total volume of material being processed. Parameters for other mixing technologies can be evaluated based on the teachings in the present disclosure.

Following completion of the application of strong mixing conditions and the formation of the composite particles, the milling particles can be removed to form a product dispersion. The product dispersion can be carried forward for additional processing into a final product, or composite particles can be separated from the product dispersion for subsequent use. The composite particles can be separated from the liquid, for example, by evaporating the liquid, using appropriately high speed centrifugation or by changing the conditions in the dispersion so that the composite particles settle from the dispersion. To settle the particles, the liquid can be mixed with a second liquid to change the properties in the dispersion.

### Toner and Ink Applications

Toner particles are deposited using electrophotography in which the particles are attracted to selectively charged portions of a surface. Toner particles have suitable properties to make them deliverable by electrophotography and for setting the toner particles into an image. The toner particles generally comprise the composite particles described herein along with a resin binder. The toner particles can incorporate additional additives if desired, such as pigments, dyes, charge moderators, waxes and the like.

The resin or polymer binder or a portion thereof can be selected to have an appropriate melting temperature for a toner particle. Suitable resins include, for example, polystyrene, polyvinyltoluene, polyesters, phenol resin, polyvinyl chloride, polyvinylacetate, polyethylene, polyurethane, epoxy resin, polyvinyl butyral, polyacrylic resin, terpene resin, aromatic petroleum resin, similar copolymers, mixtures thereof and the like. Suitable additive can adjust the electrical properties of the toner particle. Toner particles are described further, for example, in U.S. Pat. No. 6,653,037 to Sawada et al., entitled “Toner for Developing Latent Electrostatic Images, and Image Forming Method and Device,” incorporated herein by reference.

The composite particles can also be incorporated into inks for ink jet printing, lithographic printing, gravure printing, screen printing and the like. The composite particles can function as pigments and/or property modifiers to facilitate the formation of a stable image. Due to the small particle size, sharper images can be formed with less material. The use of printing inks with particulate colorants for newspaper publishing is described in U.S. Pat. No. 5,981,625 to Zou et al., entitled “Non-Rub Off Printing Inks,” incorporated herein by reference.

### EXAMPLES

### Materials

PR269 is an organic red pigment listed in C.I. (Color Index) as PR269 and is also known under various trade names such as Toshiki™ Red 1022 (Tokyo Shikizai Industry Co., Ltd), Permanent Carmine 3810 (Sanyo Color Works, Ltd.), or Red F218 (Dainichiseika Color & Chemicals Mfg. Co., Ltd.). EFKA-4080 is a commercial polymeric dispersing agent available from Ciba Specialty Chemical as a solution (proprietary composition and concentration). The TiO₂ nanoparticles were synthesized at NanoGram Corporation essentially according to the procedure described in Example 1 of U.S. patent application Ser. No. 11/645,084 to Chiruvolu et al., entitled “Composites of Polymers and Metal/Metalloid Oxide Nanoparticles and Methods for Forming These Composites,” incorporated herein by reference. The particle size and particle surface area is specified in individual examples. The modified TiO₂ particles were modified with hexamethyldisilazane (HMDZ) and polydimethoxysiloxane (PSI-026 from Gelest) as described in Example 3 of U.S. patent application Ser. No. 11/645,084. The SiO₂ nanoparticles were manufactured by Degussa GmbH. The particle size and particle surface area is specified in individual examples. The polyester resin is a polymer described in U.S. Pat. No. 4,314,049. The YTZ® milling beads and balls are yttrium stabilized zirconia milling media from Tosoh Corporation.

### Instruments

The milling experiments were done in a Thinky™ mixer—a planetary centrifugal mixer model ARE-250R. Bead milling experiments were done in Netzsch MiniCer™ bead mill. Ball milling experiments were done in US Stoneware ball mill. Dynamic light scattering measurements of dispersions were made with a Malvern Zetasizer™ Nano-ZS to evaluate secondary particle sizes. Results for dynamic light scattering (DLS) measurements are reported as an intensity average, referred to as the Z-average or cumulants mean. UV-vis measurements were done with a Scinco™ S-3100 instrument. The sonication experiments were performed with Branson 5510 ultrasonic bath. Centrifugation was performed in a Beckman Coulter Allegra™ 25R centrifuge.

### Optical Density, Haze and Color Properties Measurements

The concentration of the organic pigment in the milled paste was evaluated by dissolving an aliquot of the pigment in N-methylpyrrolidone (NMP), measuring the optical transmission of the resulting solution at 550 nm and recalculating the concentration and dilution factor of the pigment. Milled pastes were then mixed with calculated amounts of ethyl acetate (EA) and polyester resin. The formulations were cast onto PET (polyethylene terephthalate) film by the method of dragging a wire bar. The films were dried for 24 h. The color properties and haze were measured by 8400 X-rite instrument in specular excluded reflective mode. The coloring power was determined by X-rite™ instrument software as “Status T density” and was plotted against deposited weight of pigment. The deposited weight of the pigment was calculated from the predetermined concentrations of pigment, the predetermined size of the wire bar, and the predetermined film thickness deposited by the wire bar. Color coordinates for the films were measured in specular excluded mode using standard features of 8400 X-rite™ instrument. Color gamut values for composite particles were determined by plotting color coordinates for samples of various concentrations. The plots for various composite particles were overlaid and compared qualitatively to plots of blank PR269 samples. Haze values for the films were measured in transmission mode using standard features of 8400 X-rite™ instrument.

### Example 1

### Changes with the Core Material

This example demonstrates the optical properties of composite particles formed with TiO₂ or SiO₂ nanoparticles following a milling process. The Example also demonstrates that as inorganic cores for the organic pigment PR269, unmodified TiO₂ nanoparticles gave better coloring power than surface modified TiO₂ nanoparticles. SiO₂ nanoparticles were also tested as a core material in comparison to TiO₂ nanoparticles.

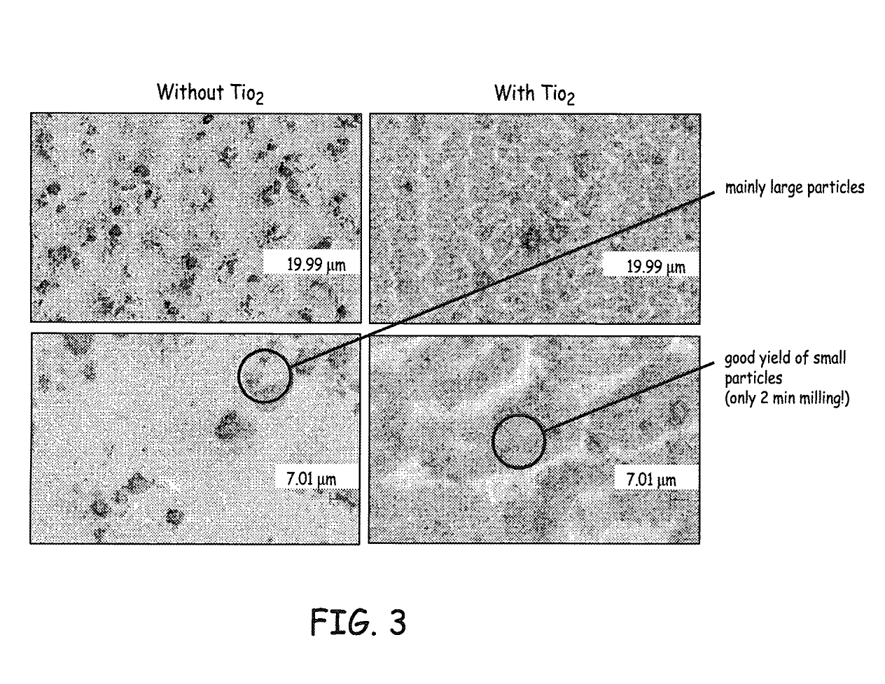

Two samples were made and tested to examine the effects of milling organic pigments in the presence of inorganic nanoparticles. Sample 1 was formed using 250 mg unmodified TiO₂ nanoparticles (surface area 48 m²/g), 250 mg PR269, 2.5 mL EFKA-4080 solution, and 2.5 mL ethyl acetate (EA). Sample 2 was formed using 250 mg PR269, 2.5 mL EFKA-4080 solution, and 2.5 mL EA without unmodified TiO₂ nanoparticles. Each sample was milled in a Thinky mixer for 2 min at 2000 RPM to produce a thick paste. Optical micrographs for the samples are shown in FIG. 3. The optical micrographs of sample 1 with the nanoparticles (two images on the right at different resolutions) illustrates that the process yielded smaller particles after milling. Sample 2 in comparison yielded mainly large particles (two images on the left at different resolutions).

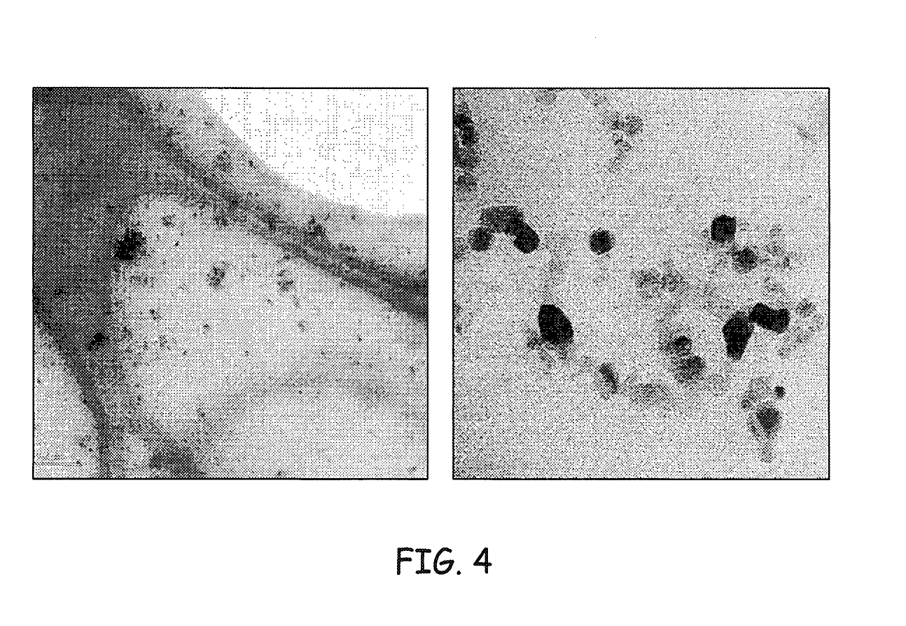

Similarly, sample 3 with 400 mg unmodified TiO₂ nanoparticles (surface area 150 m²/g), 100 mg PR269, and 3 mL EFKA-4080 solution was milled 40 min in a Thinky™ mixer at 2000 RPM to make a thick paste. Transition electron (TEM) micrographs with different resolutions of a diluted sample of the thick paste is shown in FIG. 4. The micrograph reveals good dispersion of the titanium dioxide particles with pigment. The presence of TiO₂ nanoparticles in the samples of FIGS. 3 and 4 apparently facilitated the formation of small pigment particles with pigment coated on the inorganic particles, and good dispersion.

Additional samples were milled with commercial milling beads with the exception of a control blank sample 4 that was sonicated in an ultrasonic bath for 1 h instead of milling. As shown in Table 1, samples 4-6 with the specified compositions were prepared.

TABLE 1

| | | | EFKA-4080 | | YTZ ® |
|---|---|---|---|---|---|
| Sample | PR269 | TiO₂ | solution | EA | Beads, 50 μm |
| 4 (Blank) | 0.3 g | No | 1.5 g | 3 g | None |
| 5 | 0.3 g | Yes, | 1.5 g | 2 g | 2.7 g |
| | | unmodified, | | | |
| | | 0.3 g | | | |
| 6 | 0.3 g | Yes, modified, | 1.5 g | 2 g | 2.7 g |
| | | 0.3 g | | | |

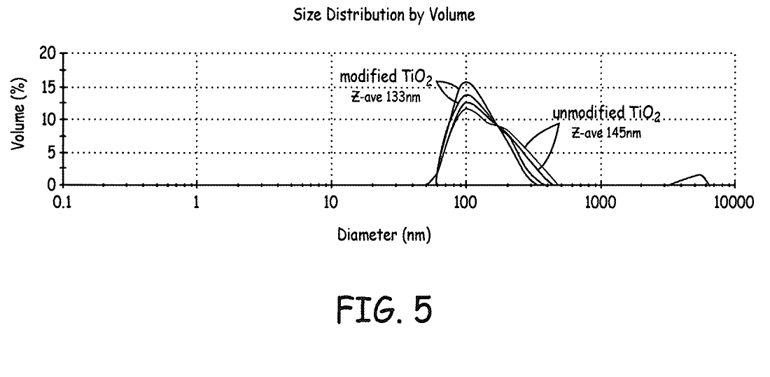

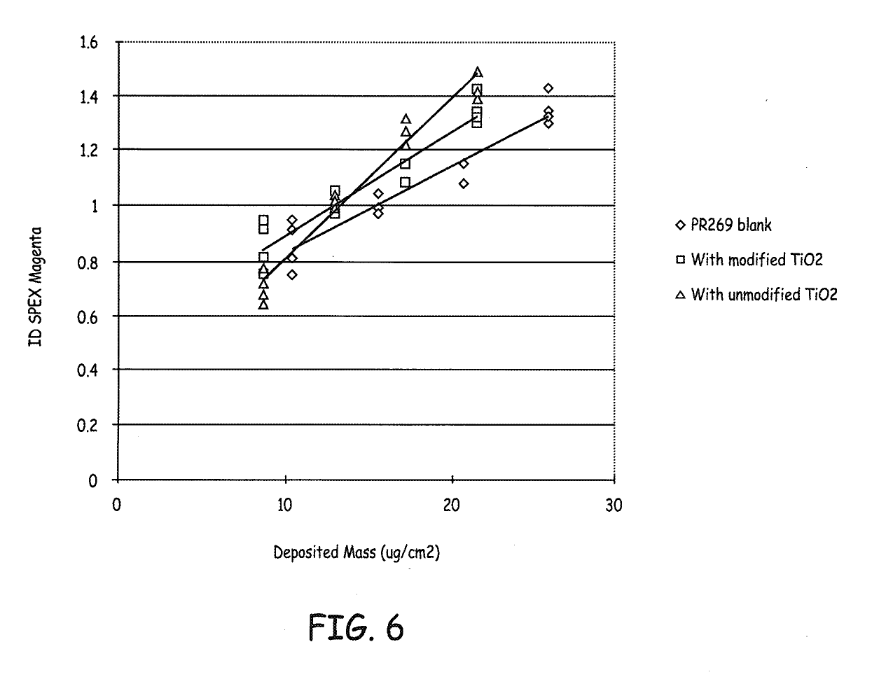

Specifically, sample 5 with unmodified TiO₂ particles and sample 6 with modified TiO₂ particles were each milled in a Thinky™ mixer for 30 min at 2000 RPM with 50 μm YTZ® milling beads. The results were evaluated by DLS. As shown in FIG. 5, the HMDZ/PSI-026 modified TiO₂ particles had a slightly smaller average diameter (133 nm) than unmodified TiO₂ particles (145 nm). Sonicated sample 4 was mixed with calculated amounts of EA and polyester resin to yield 1% PR269 and 29% polyester resin formulation. Milled samples 5 and 6 were mixed with calculated amounts of EA and polyester resin to yield 1% PR269, 1% TiO₂, and 29% polyester resin formulations. The coloring power of samples 4-6 was determined as described above, and is plotted in FIG. 6. The coloring power appeared to be higher when unmodified TiO₂ was present. Unmodified TiO₂ appeared to yield even higher coloring power than surface modified TiO₂.

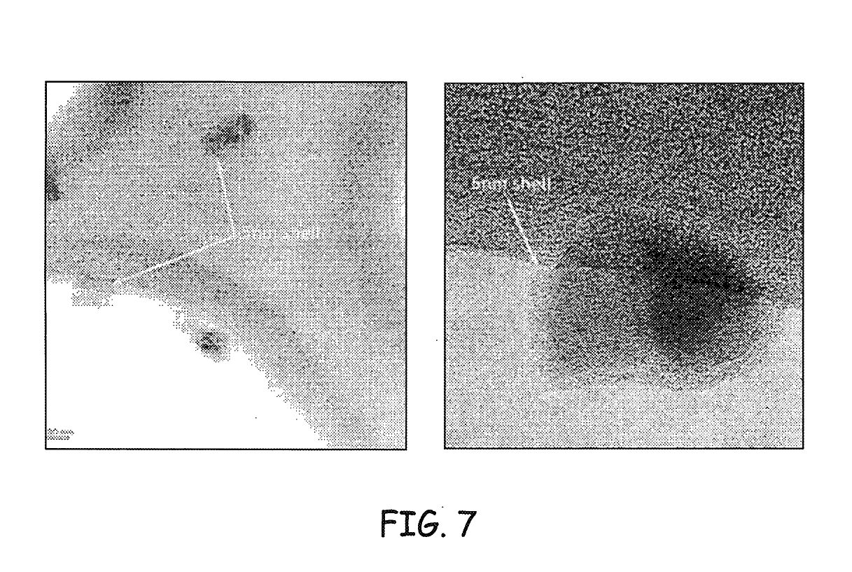

A different core material with selected particle sizes was tested as a core material in comparison to TiO₂ nanoparticles. Four samples, samples 7-10 were prepared with ball milling as described in Table 2. The samples were milled in a ball mill with 0.8 mm YTZ® beads. The thicknesses of pigment shell of the composite particles were calculated from the amounts of core and shell materials loaded. For samples 7-10 (using 20 nm SiO₂, 40 nm SiO₂, 20 nm TiO₂, and 40 nm TiO₂ as core material, respectively), pigment shell thicknesses of 6 nm, 13 nm, 6 nm, and 11 nm were calculated respectively. The particles were evaluated using TEM, and the dispersions were evaluated using DLS. As shown in FIG. 7, TEM of sample 9 (20 nm TiO₂ composite particles with 6 nm pigment shell) demonstrated the presence of TiO₂ core and organic pigment shell.

TABLE 2

| | | | EFKA-4080 | | Core | Shell | Milling | Zₐᵥₑ |
|---|---|---|---|---|---|---|---|---|
| Sample | PR269 | Core | solution | EA | diameter | thickness | time | by DLS |
| 7 | 7.3 g | SiO₂, | 19.5 g | 40 g | 20 nm | 6 nm | 43 h | 146 nm |
| | | 4 g | | | | | | |
| 8 | 8.8 g | SiO₂, | 22.8 g | 40 g | 40 nm | 13 nm | 48 h | 165 nm |
| | | 4 g | | | | | | |
| 9 | 3.8 g | TiO₂, | 10.5 g | 40 g | 20 nm | 6 nm | 88 h | 107 nm |
| | | 4 g | | | | | | |
| 10 | 3.8 g | TiO₂, | 10.5 g | 40 g | 40 nm | 11 nm | 39 h | 126 nm |
| | | 4 g | | | | | | |

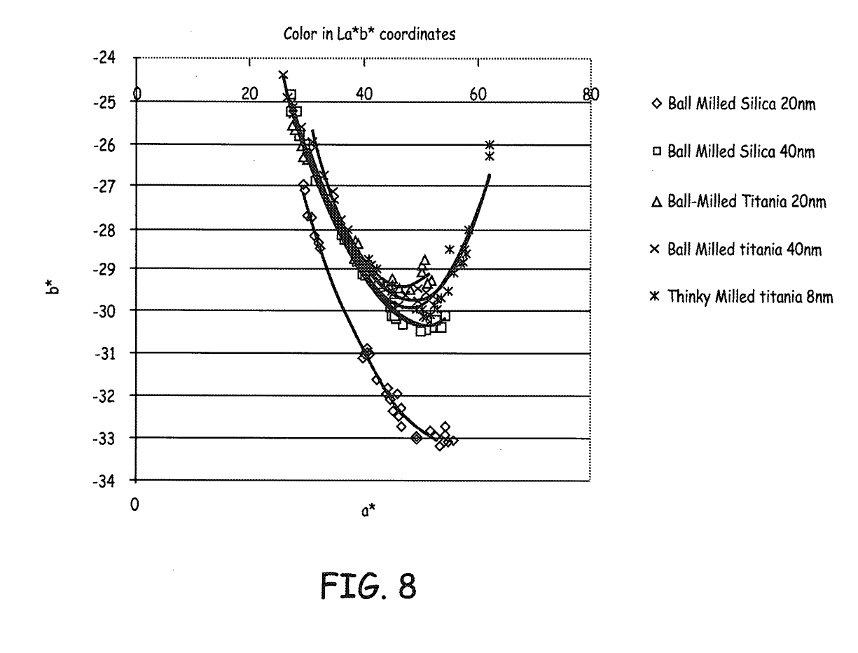

The milled pastes of sample 7-10 were mixed with EA and polyester resin to give 29% polyester resin with 1% PR269. The color gamut and haze of the samples were compared to the properties of the blank sample described above as sample 4. As shown in FIG. 8, smaller particles and low refractive index core or no core samples (20 nm SiO₂ composite particles or pure pigment blank) yielded the broadest gamut. Core-shell particles with low refractive index core (SiO₂) exhibited narrower color gamut for large cores (40 nm SiO₂ cores). Core shell particles with high refractive index core (TiO₂) exhibited narrower color gamut even for small cores. For all samples, haze was very low: 3% for 20 nm SiO₂ and TiO₂, 3-4% for 40 nm TiO₂, and 4-7% for 40 nm SiO₂.

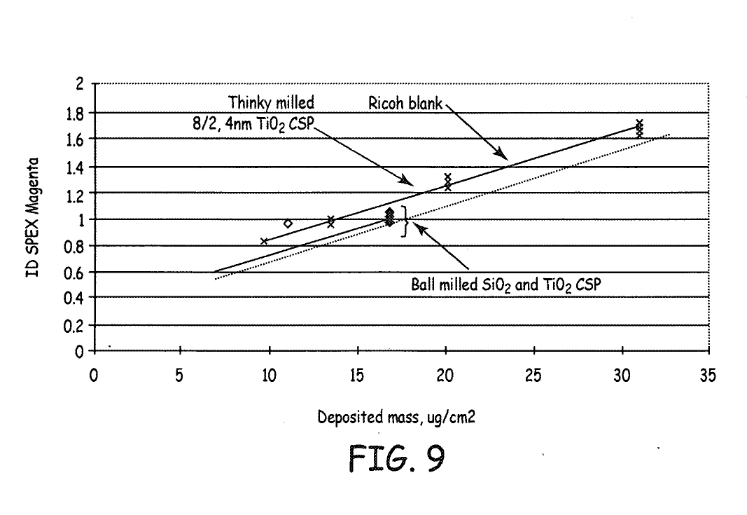

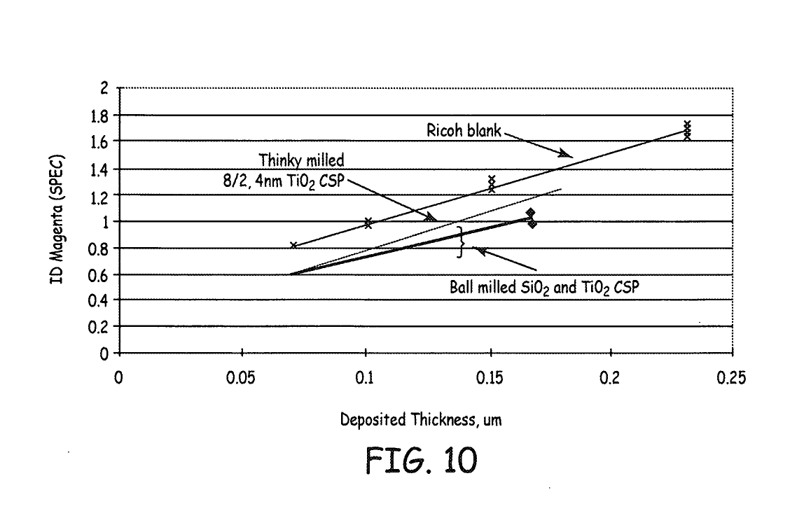

Additionally, the coloring power of the samples 7-10 was evaluated. As shown in FIG. 9, the coloring power of all samples was within margin of error of coloring of pure PR269 when plotted for deposited mass of pigment PR269 itself. There was no significant dependence of coloring power on core size, core type, or milling method. The coloring power of composite particles was weaker than pure PR269 when plotted for deposited volume of core and shell as shown in FIG. 10.

### Example 2

### Changes with the EFKA-4080 Concentration

The impact of EFKA-4080 concentration on particle size and coloring power was evaluated in this example. The EFKA-4080 concentration was calculated proportional to IPW (imaginary pigment weight), which is a weight of an imaginary particle equal in volume to the composite particle, but made entirely of PR269. The purpose of using IPW is to convert weight percent loadings into volume factors, as volumes (and surface areas) are more meaningful for surface protecting agents (such as EFKA-4080):

IPW=(V_{PR}+V_{TiO2})×d_{PR}=(m_{PR}/d_{PR}+m_{TiO2}/d_{TiO2})×d_{PR}

Where V is volume, d is density, and m is mass; d_{TiO2}=4.27, d_{PR}=1.34
Actual amount of EFKA-4080=IPW×EFKA-4080%/PR%

All the EFKA-4080 concentrations used in this application are compensated with IPW. For example, when PR%=1%, formulations 1-3 with compositions shown in Table 3 were used to prepare samples.

TABLE 3

| | EFKA- | Actual amount of EFKA-4080 per 100 mg |
|---|---|---|
| Formulations | 4080% | PR + 100 mg TiO₂ |
| F-1 | 0.67% | 88 mg |
| F-2 | 2.00% | 263 mg |
| F-3 | 4.00% | 526 mg |

All samples were milled to a thick paste with PR269, EFKA-4080, EA, non-modified TiO₂ (with surface area 180 m²/g), and 50 μm beads in a Thinky™ mixer for 30 min at 2000 RPM. To evaluate the optical properties of the resulting composite particles, the thick paste was mixed with polyester resin and diluted with EA to a final composition of 1% PR269, 1% non-modified TiO₂, and 29% resin. The films were cast, and the color property measurements were performed as described above.

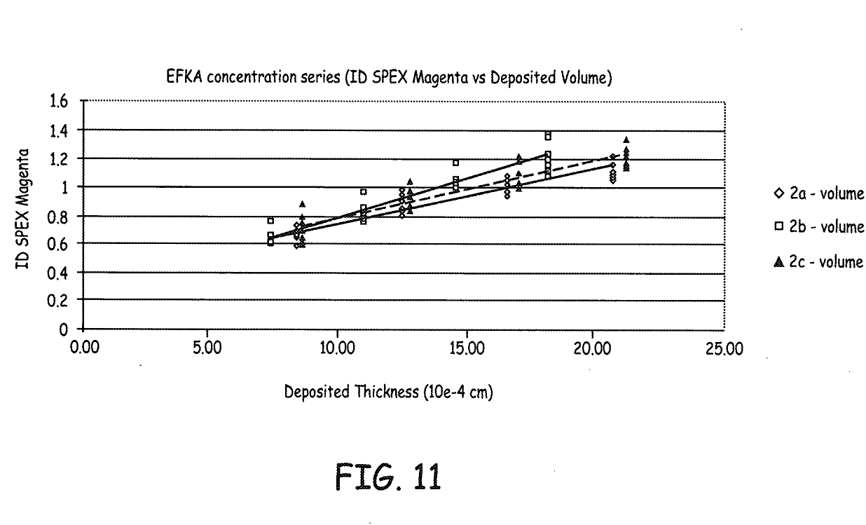

Time series (different milling time period) for different EFKA-4080 concentration was performed. Samples 11-13 with 0.67% EFKA-4080 are milled in a Thinky™ mixer for 10, 20, 30 min each. The milling yielded composite particles with average sizes of 280, 520, and 1400 nm each for the 10, 20, 30 min milling time, respectively. Particle size increased with longer milling was most likely indicative of unstable dispersion condition. The 0.67% EFKA-4080 used therefore was insufficient to achieve desired level of protection of the organic pigment shell surface with EFKA-4080 dispersing agent. Samples with 2% (samples 14-16) and 4% (samples 17-19) EFKA-4080 were also milled for 10, 20, 30 min each. The milling yielded composite particles with average sizes of 170, 150, and 140 nm for the 2% series and 160, 130, and 150 nm for the 4% series. If the same array of samples is compared at milling time of 30 min (Samples 13, 16, and 19 with 0.67%, 2% and 4% EFKA-4080) the milling yields composite particles with average sizes of 1400, 140 and 150 nm each for the 0.67%, 2% and 4% EFKA-4080 concentrations, respectively. Based on this result, 2% EFKA-4080 appeared to be sufficient to achieve desired secondary particle sizes. Coloring power of samples 13, 16 and 19 milled for 30 min with 0.67%, 2% and 4% EFKA-4080 was tested. As shown in FIG. 11, 2% EFKA-4080 appeared to give the best coloring power. For these samples, a 2% EFKA-4080 amount of pigment dispersing agent was found to be sufficient for obtaining desired secondary particle size ranges.

A scaled up experiment was performed with relatively high EFKA-4080 concentration. A mixture of 1.5 g of PR269, 1.5 g TiO₂ (surface area 170 m²/g), 3.94 g EFKA-4080 solution, 12 g EA, and 44 g of 50 μm YTZ® beads was milled for 30 min at 2000 RPM in a Thinky™ mixer to yield an average particle diameter of 170 nm. The paste was then formulated into a final sample 20 with 0.564% composite particles (0.262 TiO₂+0.282% PR269), 0.68% EFKA-4080, and 29% resin in EA.

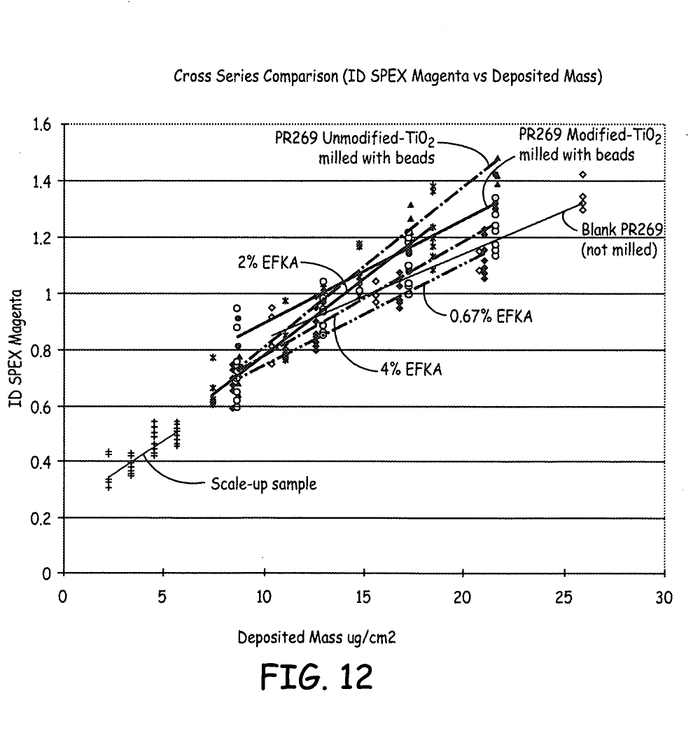

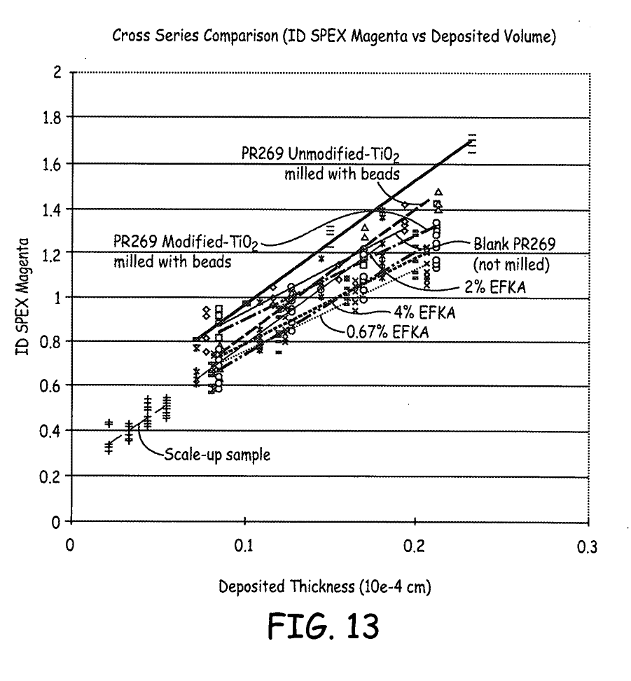

Coloring power of the samples 4-6 from Example 1 are compared with the new results obtained from samples 13, 16 and 19 in this example. As shown in FIG. 12, coloring power of composite particles appeared to be higher than that of the blank sample per deposited mass of PR269. The coloring power of the 2% EFKA-4080 sample was very close to the best samples. The coloring power of the scaled up sample 20 was also included in the graph. When composition per deposited volume of the samples was compared, good composite particle samples appear to give a markedly different slope of the image density line, as shown in FIG. 13. It needs to be considered that deposited volume of composite particle included colorless titanium dioxide particles. Plots for deposited volume therefore could have masked the increase in color strength.

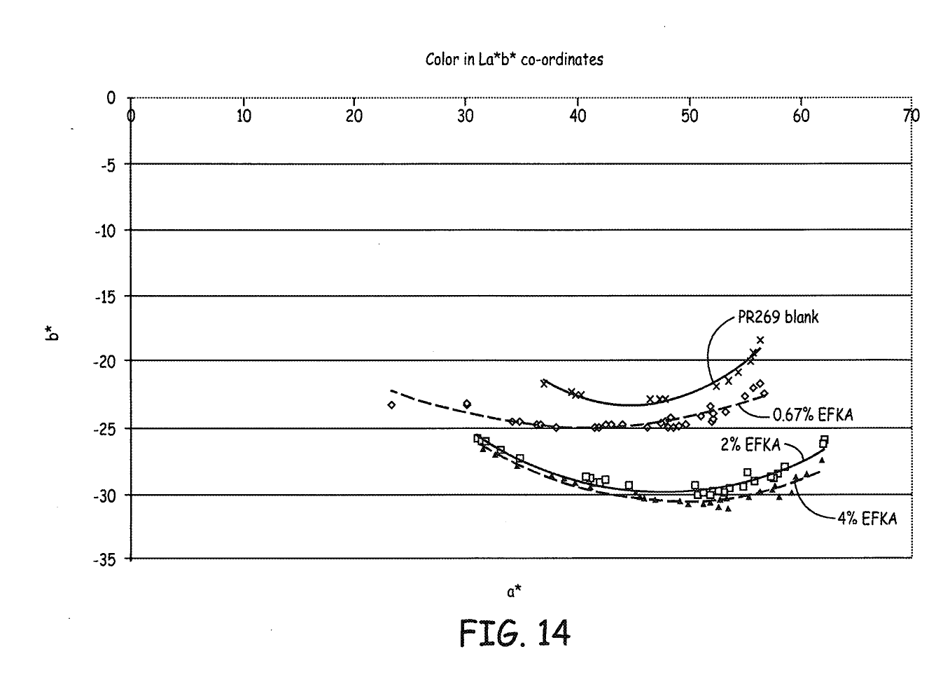

Color coordinates of samples with different EFKA-4080 concentrations are also evaluated. As shown in FIG. 14, color gamut of composite particle samples is considerably expanded compared to the regular blank sample.

In a separate experiment, samples 21-24 were prepared and milled according to the conditions specified in the Table 4.

TABLE 4

| Sample | EFKA-4080 | Milling time (min) |
|---|---|---|
| 21 | 2% | 15 |
| 22 | 2% | 30 |
| 23 | 2% | 30 |
| | and additional 2% | and additional 15 |
| 24 | 2% | 30 |
| | and additional 2% | and additional 45 |

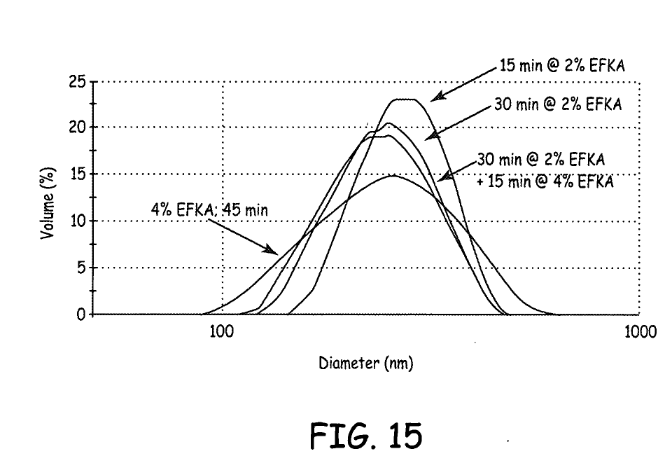

All samples were milled with 0.3 mm YTZ™ beads at 3000 RPM in a bead mill in EA with 1% PR269, 1% TiO₂ (20 nm, 50 m²/g), and with 2% or 4% EFKA-4080. As shown in DLS measurements of FIG. 15, 2% EFKA-4080 appeared to give good dispersions as determined by Z-average secondary particle sizes. Application of additional EFKA-4080 as shown by results from Sample 23 did not improve the composite particle properties, as evaluated by secondary particle size. Longer milling time as shown by the results from sample 24 appeared to broaden composite particle average diameter distribution on both ends.

### Example 3

### Changes with the PR:TiO₂ Ratio

The following experiments explore the effect of the PR:TiO₂ ratio on the properties of the resulting composite particles.

All samples were milled with PR269, EFKA-4080, non-modified TiO₂ (with surface area 180 m²/g), and just enough EA to form a thick paste (see Table 5 for exact amounts). The samples were milled with 50 μm beads in a Thinky™ mixer for 30 min at 2000 RPM.

TABLE 5

| | | | EFKA-4080% per | Actual amount of |
|---|---|---|---|---|
| Formulation | PR269 | TiO₂ | IPW | EFKA-4080 |
| F-4 | 200 mg | 200 mg | 2% | 526 mg |
| F-5 | 200 mg | 40 mg | 2% | 425 mg |
| F-6 | 200 mg | 1000 mg | 2% | 1028 mg |

The thick paste was mixed with resin and diluted with EA to a final composition of 1% PR269, 1% non-modified TiO₂, 2% EFKA-4080, and 29% resin to yield samples 25-28 shown in Table 6.

Judging from the experimental results, samples 25-27 (1:1 to 5:1 PR:TiO₂) appeared to give average diameters (140 nm, 170 nm and 160 nm for samples 25-27, respectively) and acceptable haze, whereas sample 28 (1:5 PR:TiO₂) gives significantly larger average diameter of 400 nm.

TABLE 6

| Sample | PR:TiO₂ | Haze | Diameter nm | EFKA-4080:PR |
|---|---|---|---|---|
| 25 | 1:1 ~66% beads | 5-7% | 140 | 2.63 (2:1 per IPW) |
| 26 | 1:1 ~40% beads | 4-5% | 170 | 2.63 (2:1 per IPW) |
| 27 | 5:1 ~66% beads | 4% | 160 | 2.12 (2:1 per IPW) |
| 28 | 1:5 ~66% beads | 5-8% | 400 | 5.14 (2:1 per IPW) |

### Example 4

### Changes with the Dispersion Media

This example explores the properties of the composite particles that are processed with different dispersing liquids.

Solvents ethyl acetate (EA), propylene glycol (PG), methyl ethyl ketone (MEK), methoxypropanol, acetonitrile, water, and methanol were tested as dispersion media.

Samples 29-31 with water, methanol, and EA, respectively, as the solvents were tested during the milling process. The samples were milled with 0.3 mm YTZ® beads at 3000 RPM in a Netzsch MiniCer™ bead mill with 1% PR269 and 1% TiO₂ (20 nm, 50 m²/g). Dispersion in water was milled without dispersant, whereas dispersions in EA and methanol were milled with 2% EFKA-4080. Water and methanol gave poor dispersions, whereas EA gave acceptable dispersion.

Samples 32-35 with PG, MEK, methoxypropanol, and acetonitrile, respectively, as solvent were tested in post milling stages. An 1 mL aliquot of sample 22 described above was treated with 5 mL hexanone and 5 mL hexane, and centrifuged at 6000 RPM for 5 min. The precipitate was mixed with PG, MEK, methoxypropanol, or acetonitrile, respectively, to give samples 32-35. The mixture was redispersed by 1 h of sonication in an ultrasonic bath and characterized by DLS. MEK and PG dispersions furnished smaller average particle size than parent dispersion in EA. Methoxypropanol and acetonitrile gave poor dispersions.

### Example 5

### Comparison of Milling Techniques

The effect of using beads in the milling process was examined in experiments presented in this example.

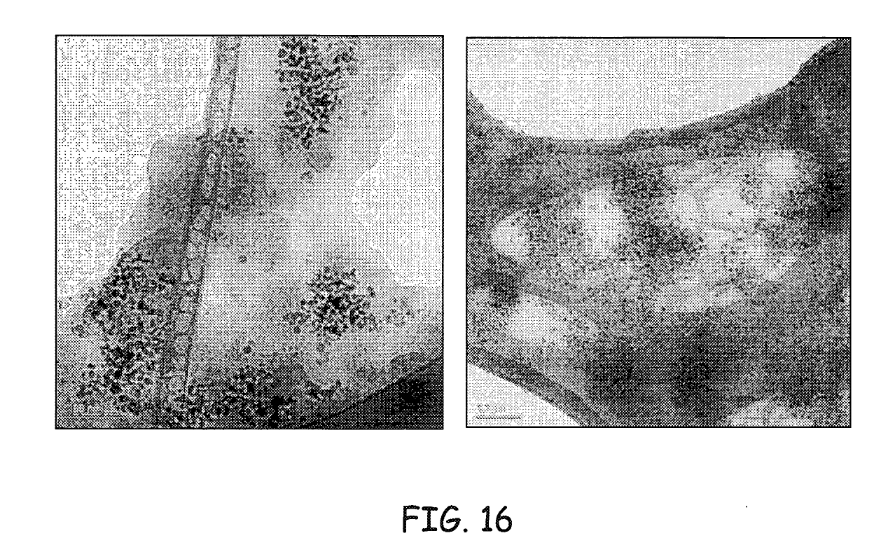

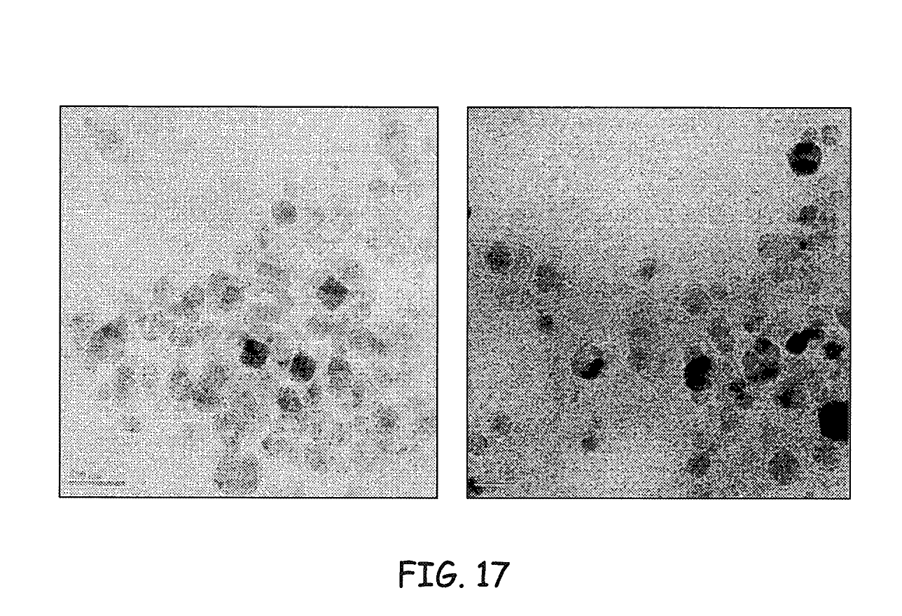

Samples with a PR269:TiO₂:EFKA-4080 ratio of 1:4:5 were milled for 4 min in a Thinky mixer with 50 μm beads (sample 36) and without beads (sample 37). The milled particles were then examined by TEM. The left image in FIG. 16 was taken from sample 36. The right image was taken from sample 37. Apparently, sample 37 had better dispersion than sample 36. Similarly in FIG. 17, the right image shows sample 37, and the left image shows the sample 36 at a different magnification than the images taken of FIG. 16

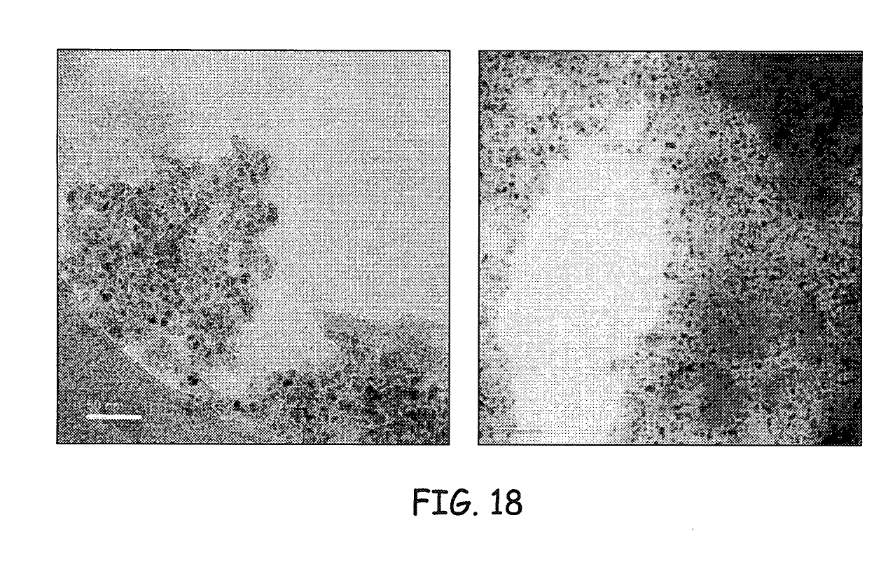

Also, sample 38 with PR269:TiO₂:EFKA-4080 in a ratio of 1:1:10 was milled with 50 μm beads in a Thinky mixer for 40 min. The image of the sample was shown in the right micrograph of FIG. 18. The left micrograph in FIG. 18 was taken from the sample which was milled without beads and EFKA-4080 for 15 min in a Thinky™ mixer (PR269:TiO₂ ratio of 1:4, sample 39). Sample 39 was then modified by adding EFKA-4080 to obtain a PR269:TiO₂:EFKA-4080 ratio of 1:4:5 and milled for an additional 15 min. Comparing the two images in FIG. 18, the right image from the sample milled with 50 μm beads appeared to have very good dispersion.

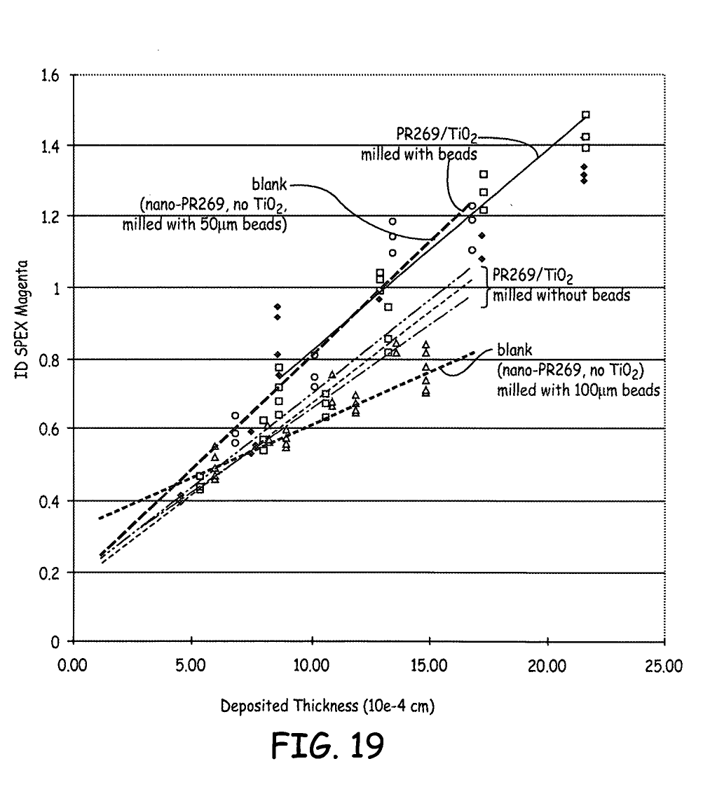

Blanks 1 (sample 40), 2 (sample 41) and samples 42-47 with the compositions shown in Table 7 were prepared and tested. The blank samples were prepared by sonication in an ultrasonic bath for 1 h. The rest of the samples were prepared by milling a 1:1 mixture of PR269 and TiO₂ (surface area 180 m²/g) in a Thinky mixer. The test results were plotted in FIG. 19. As shown in FIG. 19, the coloring power of the pigments onto the nanoparticles improved with milling. With respect to obtaining a smaller Z-average secondary particle size for the composite particles, milling with TiO₂worked better than milling with 100 μm beads alone, milling with TiO₂worked almost as good as with 50 μm beads alone, and milling with TiO₂+beads worked better than milling with TiO₂ alone.

TABLE 7

| | | | | Average composite |
|---|---|---|---|---|
| Sample | PR269 | TiO₂ | Bead | particle size |
| 40 (Blank 1) | Yes | No | 50 μm | 180 |
| 41 (Blank 2) | Yes | No | 100 μm | 220 |
| 42 | Yes | Yes | No | 180 |
| 43 | Yes | Yes | No | 190 |
| 44 | Yes | Yes | No | 200 |
| 45 | Yes | Yes | No | 230 |
| 46 | Yes | Yes | 50 μm | 260 |
| 47 | Yes | Yes | 100 μm | 250 |

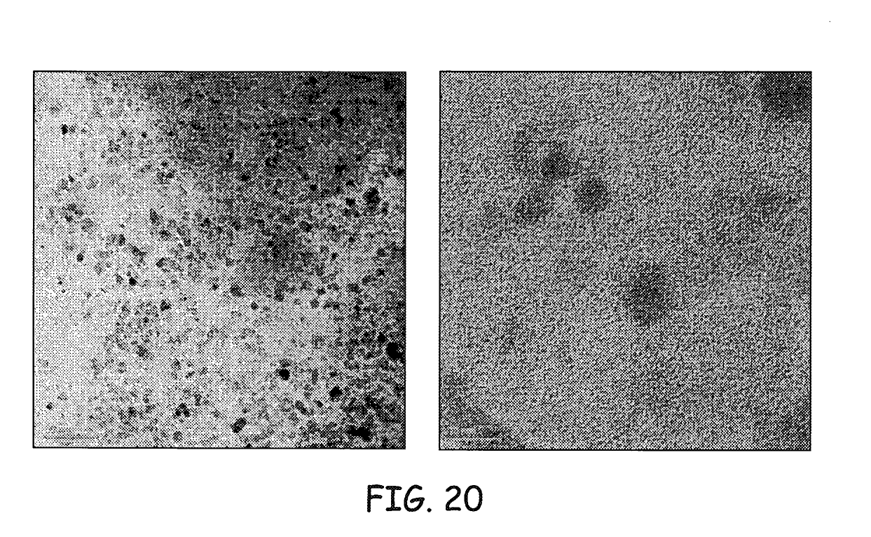

Sample 48 with a PR269:TiO₂:EFKA-4080 ratio of 1:1:10 (TiO₂ surface area 180 m²/g) was milled with 50 μm beads in a Thinky™ mixer for 40 minutes. The image of the sample was shown in the micrographs of FIG. 20. The left micrograph showed the breakdown of loose agglomerates of TiO₂, reduction in particle size of the pigment, and a well dispersed mixture particles. The right micrograph was an enlarged picture of a portion of the left micrograph.

Bead mill, ball mill and Thinky™ mixer were tested and the milling results compared to choose the most efficient and scalable milling technique to improve particle size and dispersion quality as well as to establish good base-line milling procedure. A series of samples was milled in a US Stoneware ball mill. Each sample was a mixture of 1 g PR269, 1 g TiO₂(20 nm, surface area 50 m²/g), 10.5 g EFKA-4080, and 40 mL EA. Another series of samples was milled in a Netzsch MiniCer™ bead mill. Each sample was a mixture of 2 g PR269, 2 g TiO₂ (20 nm, surface area 50 m²/g), 10.5 g EFKA-4080, and 200 mL EA.

Milling speed in revolutions per minute (RPM) was tested for ball mill and bead mill. 20, 40, 70, 100, 150, and 200 RPM were tested for ball mill. The efficiency of particle size reduction was followed up by DLS measurements. Particle size reduction at 150 RPM appeared to be the best for ball mill. 1500, 2000, 2500, and 3000 RPM were tested for bead mill. The efficiency of particle size reduction was evaluated by DLS measurements. Particle size reduction at 2000 RPM appeared to be the best for bead mill.

Size of the milling media (beads) was tested for ball mill and bead mill. 5, 0.8 and 0.3 mm YTZ® beads were tested for ball mill. The efficiency of particle size reduction was followed up by DLS measurements. Particle size reduction with 0.8 mm beads appeared to be best for ball mill. 300 and 100 μm YTZ® beads were tested for bead mill. The efficiency of particle size reduction was followed up by DLS measurements. Particle size reduction with 100 μm beads appeared to be best for bead mill.

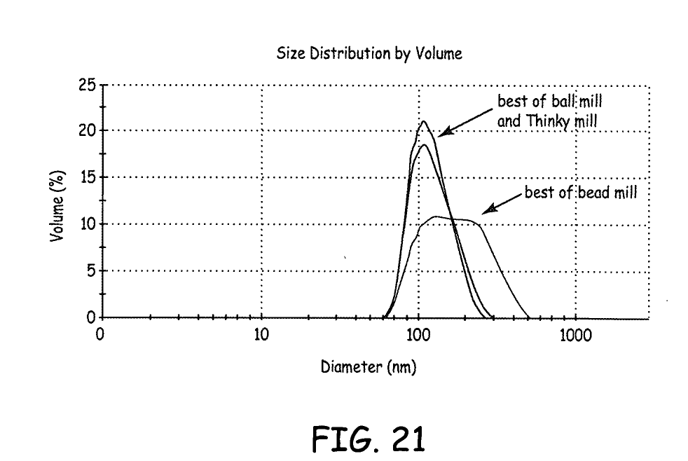

The selected best results of the ball mill, Thinky™ mixer, and bead mill milling series are shown in FIG. 21. Ball mill and Thinky™ mixer appeared to have comparable efficiency.

The embodiments above are intended to be illustrative and not limiting. Additional embodiments are within the claims. In addition, although the present invention has been described with reference to particular embodiments, those skilled in the art will recognize that changes can be made in form and detail without departing from the spirit and scope of the invention. Any incorporation by reference of documents above is limited such that no subject matter is incorporated that is contrary to the explicit disclosure herein.

What we claim is:

1. A dispersion of particles comprising a carrier liquid, a pigment dispersing agent and composite pigment particles, the composite pigment particles comprising a blend of an inorganic particle and an organic pigment, the composite pigment particles having a Z-average dispersed particle size in the organic carrier liquid of no more than about 300 nm.

2. The dispersion of claim 1 wherein the composite particles are substantially free of surface modification composition on the surface of the inorganic particles between the inorganic particle surface and the organic pigment.

3. The dispersion of claim 1 wherein the dispersion comprise from about 10 weight percent to about 500 weight percent pigment dispersing agent relative to the weight of the composite pigment particles.

4. The dispersion of claim 1 wherein the composite pigment particles comprise from about 15 weight percent to about 200 weight percent organic pigment relative to the weight of inorganic particles.

5. The dispersion of claim 1 wherein the inorganic particles comprise a metal oxide or metalloid oxide and the inorganic particles have an average particle size of no more than about 100 nm.

6. The dispersion of claim 1 wherein the composite particles have a Z-average particle size of no more than about 200 nm.

7. The dispersion of claim 1 wherein the composite particles in the dispersion have a DLS particle size distribution by volume with a width at half-height of no more than about 100 nm.

8. The dispersion of claim 1 wherein the carrier liquid is an organic solvent.

9. The dispersion of claim 1 wherein the carrier liquid comprises an organic compound selected from the group consisting of a liquid alcohol, ethyl acetate, and mixtures thereof.

10. A collection of composite particles comprising inorganic core particles and an organic pigment wherein the composite particles have an average diameter of no more than about 300 nm, wherein the inorganic core particles are substantially free of a surface modifying agent bonded between the inorganic core particles and the organic pigment and wherein the composite particles comprise at least about 5 weight percent organic pigment.

11. The collection of composite particles of claim 10 wherein the composite pigment particles comprise from about 15 weight percent to about 200 weight percent organic pigment relative to the weight of inorganic particles.

12. The collection of composite particles of claim 10 wherein the particles can be dispersed to have a Z-average particle size of no more than about 300 nm.

13. A method for forming composite pigment particles comprising applying effective mixing conditions to a blend of inorganic particles and organic pigment in an organic carrier liquid with a pigment dispersing agent to form dispersed composite particles with a Z-average particle size of no more than about 300 nm.

14. The method of claim 13 wherein the pigment dispersing agent is present in an amount from about 10 weight percent to about 500 weight percent pigment dispersing agent relative to the weight of the combined weight of inorganic particles and organic pigment.

15. The method of claim 13 wherein pigment dispersing agent comprises a polymer pigment dispersing agent.

16. The method of claim 13 wherein applying effective mixing conditions comprises milling with milling media.

17. The method of claim 16 wherein the milling media comprises inorganic beads having an average particle size from about 10 microns to about 10,000 microns.

18. The method of claim 13 wherein the inorganic particles have an average particle diameter of no more than about 200 nm and wherein the applying of effective mixing conditions is performed free of milling media.

19. The method of claim 13 wherein the composite particles have a Z-average particle size of no more than 200 nm.

20. A method for forming composite particles from an organic composition and inorganic nanoparticles in a carrier liquid wherein the organic composition and the inorganic particles are substantially insoluble in the carrier liquid, the method comprising applying effective mixing conditions to a combination of the organic composition, a dispersing agent and the inorganic nanoparticles in the carrier liquid without the presence of milling beads, to mill the organic composition onto the inorganic particles to form composite particles having an average particle size of no more than about 250 nm and wherein the inorganic nanoparticles have an average particle size of no more than about 200 nm.

21. The method of claim 20 wherein the organic composition comprises a pharmaceutical composition.

22. The method of claim 20 wherein the organic composition is a pigment.

23. A collection of composite particles comprising non-toxic inorganic particles coated with a pharmaceutical composition, wherein the inorganic nanoparticles have an average particle size of no more than about 250 nm.

24. The collection of composite particles of claim 23 wherein the inorganic particles comprise silica.
